(19) United States
(12) Patent Application Publication
(10) Pub. No.: US 2016/0237391 A1
(43) Pub. Date: Aug. 18, 2016

(54) A PROCESS FOR CHEMICAL AND/OR BIOLOGICAL TRANSFORMATION

(71) Applicant: SPINCHEM AB, Umeå (SE)

(72) Inventors: Emil BYSTRÖM, UMEÅ (SE); Henrik SCHERMAN, UMEÅ (SE); Uwe BORNSCHEUER, GREIFSWALD (DE); Knut IRGUM, BULLMARK (SE)

(21) Appl. No.: 15/026,408

(22) PCT Filed: Sep. 26, 2014
(86) PCT No.: PCT/SE2014/051113

§ 371 Date: Mar. 31, 2016

(30) Foreign Application Priority Data
Oct. 3, 2013 (SE) 1351168-8

(51) Int. Cl. C12M 3/06; C12M 1/12; C12P 7/22; C12N 11/10; C12P 17/08; C12P 13/00

(52) U.S. Cl. CPC C12M 27/14; C12P 17/08; C12P 13/001; C12P 7/22; C12N 11/10; C12M 25/16; B01F 7/28

(57) ABSTRACT

The invention relates to a process for chemical and/or biological transformation of at least one starting material dissolved in a liquid phase using at least one immobilized enzyme, cell fragments, and/or encapsulated whole cell microorganism trapped in a rotating flow distributor having an inlet for receiving liquid phase comprising starting material as well as immobilized enzyme(s)/encapsulated cell(s), a cavity for trapping said immobilized enzyme(s), cell fragment(s), and/or encapsulated whole cell microorganism(s), and outlet openings on the rotating periphery of the flow distributor.

The present invention relates to a novel process for chemical and/or biological transformation using immobilized enzymes, cell fragments, and/or encapsulated whole cell microorganisms. More specifically, the invention relates to a process for chemical and/or biological transformation of at least one starting material dissolved in a liquid phase using at least one immobilized enzyme, cell fragment fraction, and/or encapsulated whole cell microorganism trapped in a rotating flow distributor. The flow distributor has an inlet for receiving liquid phase comprising starting material as well as immobilized enzyme(s)/encapsulated cell(s), a cavity for trapping said immobilized enzyme(s), cell fragments, and/or encapsulated whole cell microorganism(s), and outlet openings on the rotating peripheral wall of the device. The invention also provides uses of such a flow distributor as well as a transformation device comprising a flow distributor and a rotation means in such a process.

### TECHNICAL BACKGROUND

Biocatalysts represents nowadays an established technology for the enzymatic synthesis of chiral building blocks for organic and pharmaceutical synthesis, compounds for the flavor and fragrance industry, production of bulk chemicals and the modification of lipids for the food industry (Breuer et al., Angew. Chem. 2004, vol. 116, p. 806; Buchholz et al., Biocatalysts and Enzyme Technology, 2^{nd} ed. Wiley-VCH, Weinheim, 2012; May et al. (Eds.), Enzyme catalysts in Organic Synthesis, Vol. 1-3, 3^{rd} ed., Wiley-VCH, Weinheim, 2012; Wenda et al., Green Chem. 2011, vol. 13, p. 3007; Liese et al. (Eds.), Industrial Biotransformations, 2^{nd} ed., Wiley-VCH, Weinheim, 2006).

Especially in combination with novel methods for enzyme discovery and protein engineering (Bornscheuer et al., Nature 2012, vol. 485, p. 185), biocatalysts became highly competitive to classical (asymmetric) chemical routes using transition metal catalysts as recently shown for the synthesis of the drug Sitagliptin (Savile et al., Science 2010, v.329, p. 305; Desai, Angew. Chem. 2011, vol. 123, p. 2018). The cost effective application of enzymes, especially for cheap products, requires immobilization of the biocatalyst (or encapsulation of whole cells or fragments thereof) to enhance their long-term stability (Mateo et al., Enzyme Microb. Technol. 2007, vol. 40, p. 1451; Iyer et al., Process Biochem. 2008, vol. 43, p. 1019), and facilitate their re-use. At the same time such cost effective application should enable use of established reactor setups such as fixed bed reactors instead of simple stirred tank reactors (Hills, Eur. J. Lipid Sci. Technol. 2003, vol. 105, p. 601). Fixed bed reactors are used, for instance for the large-scale production of chiral amines (Balkenhohl et al., J. Prakt. Chem. 1997, vol. 339, p. 381) or of emollient esters for the cosmetic sector using lipase technology. Disadvantages encountered with fixed bed reactors are however (depending on e.g. length, diameter, particle size, flow rate) the pressure drop occurring within the column, reactant and pH gradients as well as inactivation profiles after extended use. In contrast, the more simple use of a stirred tank reactor is encountered with mechanical challenges of the carrier resulting in abrasion of the biocatalyst material and severe damage of encapsulated whole cells or cell fragments, beside the fact that recycling of the immobilized biocatalyst is rather laborious.

An alternative possible setup for performing a chemical and/or biological transformation in fluid media is disclosed in WO 2011/098570. The different rotating transformation devices disclosed therein enable simultaneous stirring and efficient percolation of liquid through packed particle beds. The devices have an inlet for receiving a liquid phase comprising starting material as well as solid members capable of inducing transformation, where the inlet is located in proximity to the center of rotation, a single cavity or multiple sectorized cavities for trapping said solid members, and outlet openings on the rotating periphery of the device. However, such devices have not been used and/or described in transformation processes where biocatalysts such as immobilized enzymes, cell fragments, and encapsulated whole cell microorganisms are involved.

### SUMMARY OF THE INVENTION

The present invention solves the above mentioned problems by providing a process for chemical and/or biological transformation of at least one starting material dissolved in a fluid medium comprising the steps of:

a) providing a fluid medium containing a dissolved starting material;
b) providing carriers comprising at least one biochemical transformation means selected from the group of an immobilized enzyme, cell fragments, and an encapsulated whole cell microorganism;
c) providing a reactor vessel, in which reactor vessel a transformation device has been mounted, said transformation device comprising
a flow distributor having an essentially cylindrical shape, a first essentially flat surface, a second essentially flat surface, and a peripheral wall having an essentially cylindrical cross-section, at least one fluid medium inlet for receiving fluid medium and carriers located at the centre of said second surface, at least one fluid medium outlet permeable for said fluid medium but impermeable for said carriers, said outlet being located on said peripheral surface, a driving shaft on said first surface for enabling rotation or oscillation of the flow distributor, and at least one confinement wherein said carriers can be trapped and said transformation is performed; and
a means for rotating and/or oscillating the device;
   d) adding the fluid medium of step a) and the carriers of step b) to the reactor vessel of step c);
   e) rotating said flow distributor using said means at such a rotational speed or oscillatory rotational motion that said fluid medium of step a) and said carriers of step b) are sucked through said at least one fluid medium inlet into said at least one confinement, and that said fluid medium is transported out from the flow distributor through said at least one outlet while said carriers remain in said at least one confinement; and
   f) maintaining rotational motion of said flow distributor until said transformation is deemed to be completed.

### BRIEF DESCRIPTION OF THE ENCLOSED FIGURES

The present invention will be disclosed with reference to the enclosed figures in which:

FIG. 1 discloses a side view of an example of a transformation device comprising a flow distributor and associated means for rotation that can be used in a process in accordance with the present invention;

FIG. 2 shows a view of the second surface of the flow distributor shown in FIG. 1;

FIG. 3 discloses a cross-sectional view of the flow distributor shown in FIG. 1 along the line A-B;

FIG. 4 presents an overview of a reactor set-up comprising a transformation device as shown in FIG. 1; and

FIG. 5 outlines the biocatalytic reactions of the processes described in the experimental section.

### DETAILED DESCRIPTION OF THE INVENTION

In a first aspect, the present invention provides a process for chemical and/or biological transformation of at least one starting material dissolved in a fluid medium comprising the steps of:

a) providing a fluid medium containing a dissolved starting material;
b) providing carriers comprising at least one biochemical transformation means selected from the group of an immobilized enzyme, cell fragments, and an encapsulated whole cell microorganism;
c) providing a reactor vessel, in which reactor vessel a transformation device has been mounted, said transformation device comprising
a flow distributor having an essentially cylindrical shape, a first essentially flat surface, a second essentially flat surface, and a peripheral wall having an essentially circular cross-section, at least one fluid medium inlet for receiving fluid medium and carriers located at the centre of said second surface, at least one fluid medium outlet permeable for said fluid medium but impermeable for said carriers, said outlet being located on said peripheral wall, a driving shaft located on said first surface for enabling rotation or oscillation of the flow distributor, and at least one confinement wherein said carriers can be trapped and said transformation is performed; and
a means for rotating and/or oscillating the device;
   d) adding the fluid medium of step a) and the carriers of step b) to the reactor vessel of step c);
   e) rotating said flow distributor using said means at such a rotational speed or oscillatory rotary motion that said fluid medium of step a) and said carriers of step b) are sucked through said at least one fluid medium inlet into said at least one confinement, and that said fluid medium is transported out from the flow distributor through said at least one outlet while said carriers remain in said at least one confinement; and
   f) maintaining rotatory motion of said flow distributor until said transformation is deemed to be completed.

It should be noted that the flow distributor could be arranged in any orientation in the reaction vessel. Accordingly the first surface of a flow distributor arranged in a reaction vessel could be located on top of the flow distributor, at the bottom of the flow distributor. The first surface may also be facing the reactor vessel wall.

As disclosed herein, the term(s) “chemical and/or biological transformation” relates chemical and/or biological transformations such as chemical and/or biological reactions that can be carried out using at least one biochemical transformation means selected from the group of an immobilized enzyme, cell fragments, and an encapsulated whole cell microorganism.

As disclosed herein, the term “starting material” relates to a particular chemical compound or mixture of compounds that is/are to be transformed by the chemical and/or biological transformation.

As disclosed herein, the term “fluid medium” relates to the liquid phase in which the starting material is dissolved. Typically, the constituents of the medium are not transformed by the transformation process. Typical examples of constituents of a fluid medium are water and/or an organic solvent, buffering compounds, and stabilizing compounds. The skilled person knows how to compose a suitable fluid medium for a particular transformation.

As disclosed herein, the term “carrier” relates to any kind of solid support on to which an enzyme may be immobilized in a functional state or in which a whole cell microorganism or fragments thereof may be encapsulated. Enzyme immobilization and encapsulation of cell fragments or whole cell microorganisms are well established techniques and the skilled person knows how to choose a suitable enzyme/organism and immobilization and/or encapsulation method for a given transformation.

As disclosed herein, the term “reactor vessel” typically relates to a tank for transformation in batch. The reactor may comprise means for adding starting material, fluid medium, inert gases such as nitrogen, group 18 noble gases, or a reactant gases such as oxygen, hydrogen, or any other gaseous reactant. It may also comprise means for removing the reactor content and a reflux cooler.

As disclosed herein, the wording “outlet permeable for fluid medium but impermeable for said carriers” is intended to encompass two different variants. In a first variant, the outlet openings are smaller than the carriers and the carriers are therefore blocked from exiting. In a second variant where the original outlet openings of a particular flow distributor are larger than the carriers, the flow distributor may be furnished with an inner filter or liquid-permeable film lining the inside of the peripheral wall thereby reducing the openings.

As disclosed herein, the term “means for rotating” relates to any suitable means that may exercise rotational force or oscillatory rotational motion on the driving shaft, such as an electrical motor. The motor may be directly connected to the driving shaft, or indirectly by using a set of magnets.

In a preferred embodiment, said flow distributor comprises a plurality of separate confinements defined by separating walls.

In a preferred embodiment, the process further comprises the steps of g) removing said fluid medium from said reaction vessel while maintaining rotation of said flow distributor at said minimum rotation speed, thereby draining said flow distributor while said carriers are maintained inside said confinement.

In a preferred embodiment, said carriers are alginate beads encapsulating whole cell microorganisms or fragments thereof.

In a preferred embodiment, said fluid medium comprises calcium chloride, and an alginate suspension of whole cell microorganisms or fragments thereof are injected into said fluid medium during step d).

Alternative means for entrapment of whole cell microorganisms or fragments thereof are different native polysaccharides such as straight and branched celluloses, starches, dextrans, agar/agarose, carrageenans, gellan, welan, and xanthan gums, pectins, and chitin/chitosan, and alkylated, acetylated, or glycidylated derivatives thereof; proteins such as collagen, gelatin, and albumin; synthetic polymer gels such as crosslinked poly(acrylamide), polysiloxanes; thermosresponsive polymers such as poly(N-isopropyl acrylamide), poly(vinyl caprolactam) and poly(vinyl methyl ether), sol-gel derived carriers prepared by hydrolysis and polycondensation of tetraalkoxysilanes, and porous inorganic carriers such as silica. The skilled person is well aware of suitable entrapment media and may choose a suitable material and entrapment process for a given situation.

In a preferred embodiment, said whole cell microorganisms or fragments thereof are integral or fragmented bacteria and/or yeast.

In a preferred embodiment, said microorganisms are chosen from the group of the genera Acetobacter, Achromobacter, Acidovorax, Acinetobacter, Acremonium, Agrobacterium, Alcaligenes, Amycolatopsis, Arthrobacter, Aspergillus, Aureobacterium, Aureobasidium, Bacillus, Beauveria, Brevibacterium, Burkholderia, Caldariomyces, Candida, Chromobacterium, Clonostachys, Clostridia, Comamonas, Coprinus, Corynebacterium, Corynesporium, Cryptococcus, Curvularia, Enterobacter, Erwinia, Escherichia, Fusarium, Geotrichum, Gluconobacter, Gordonae, Haloferax, Helminthosporium, Humicola, Klebsiella, Kluyveromyces, Lactobacillus, Leptoxyphium, Leuconostoc, Microbacterium, Mortierella, Mucor, Mycobacterium, Neurospora, Nocardia, Ochrobactrum, Penicillium, Pichia, Plantomycetes, Protaminobacter, Pseudomonas, Pyrococcus, Rhizopus, Rhodococcus, Rhodosporidium, Rhodotorula, Rubiginosus, Saccharomyces, Serratia, Shigella, Spirulina, Staphylococcus, Stenotrophomonas, Streptomyces, Sulfolobus, Thermoactinomyces, Thermoanaerobacter, Thermoanaerobium, Thermobifida, Thermomyces, Thermus, Trigonopsis, Vibrio, Yarrowia, Zygosaccharomyces, and Zymomonas, or combinations thereof. Cells trapped and utilized according to the method disclosed in the present invention could also be derived from plants (for instance Arabidopsis, Hevea, Geranium, or Prunus) or animals, including humans.

In a preferred embodiment, the biochemical transformation means is an immobilized enzyme selected from the group of oxidoreductases, transferases, hydrolases, lyases, isomerases, and ligases.

Examples of carrier materials include different native polysaccharides such as straight and branched celluloses, starches dextrans, agar/agarose, carrageenans, gellan, wellan, and xanthan gums, pectins, and chitin/chitosan, and alkylated, acetylated, or glycidylated derivates thereof, proteins such as collagen, gelatin, and albumin, synthetic polymer gels such as cross-linked poly(acrylamide), polysiloxanes, thermosresponsive polymers such as poly(N-isopropyl acrylamide), poly(vinyl caprolactam) and poly(vinyl methyl ether), sol-gel-derived carriers prepared by hydrolysis and polycondensation of tetraalkoxysilanes, polystyrene, polyacrylates, polymethacrylates, polyamides, poly(vinyl azlactone), vinyl and allyl polymers, benthonite, zeolite, diatomaceous earth, carbon, silica, glass (non-porous and controlled pore), metals, and controlled pore metal such as alumina, zirconia and titania. The skilled person is well aware of suitable carrier materials and may choose a suitable material for a given situation.

In a second aspect, the present invention provides use of a transformation device comprising
a flow distributor having an essentially cylindrical shape, a first essentially flat surface, a second essentially flat surface, and a peripheral wall having an essentially circular cross-section, at least one fluid medium inlet for receiving fluid medium and carriers located at the centre of said second surface, at least one fluid medium outlet permeable for said fluid medium but impermeable for said carriers, said outlet being located on said peripheral wall, a driving shaft located at said first surface for enabling rotation or oscillation of the flow distributor, and at least one confinement wherein said carriers can be trapped and said transformation is performed; and
a means for rotating and/or oscillating the device;
   in a process in accordance with said first aspect.

In a preferred embodiment, said flow distributor comprises a plurality of separate confinements defined by separating walls.

In a third aspect, the present invention provides use of a flow distributor having an essentially cylindrical shape, a first essentially flat surface, a second essentially flat surface, and a peripheral wall having an essentially circular cross-section, at least one fluid medium inlet for receiving fluid medium and carriers located at the centre of said second surface, at least one fluid medium outlet permeable for said fluid medium but impermeable for said carriers, said outlet being located on said peripheral wall, a driving shaft located on the first surface for enabling rotation or oscillation of the flow distributor, and at least one confinement wherein said carriers can be trapped and said transformation is performed, in a process in accordance with the first aspect.

In a preferred embodiment, said flow distributor comprises a plurality of separate confinements defined by separating walls.

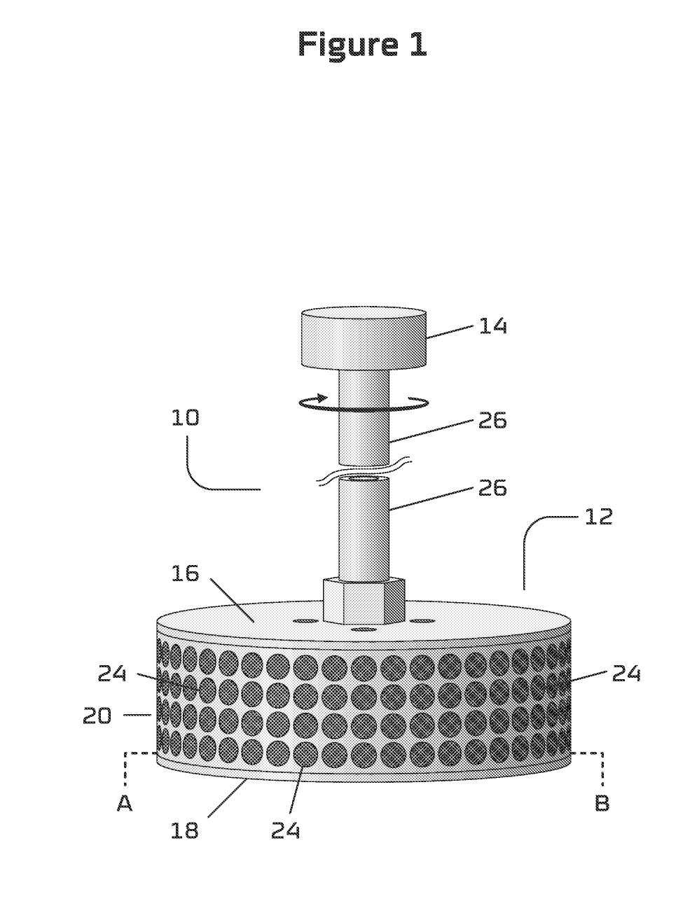

The present invention will now be further described with reference to the enclosed FIGS. 1-4.

FIG. 1 presents a side view of a transformation comprising a flow distributor 12 and a rotation means 14. The flow distributor has a first surface 16, a second surface 18 and a peripheral wall 20. The flow distributor 12 has an essentially cylindrical shape and the peripheral wall 20 has an essentially circular cross-section. Centrally on top of the first surface 16 is a drive shaft 26 for rotating and/or oscillating the flow distributor 12. This drive shaft is connected to a rotation and/or oscillation means 14, typically an electrical motor.

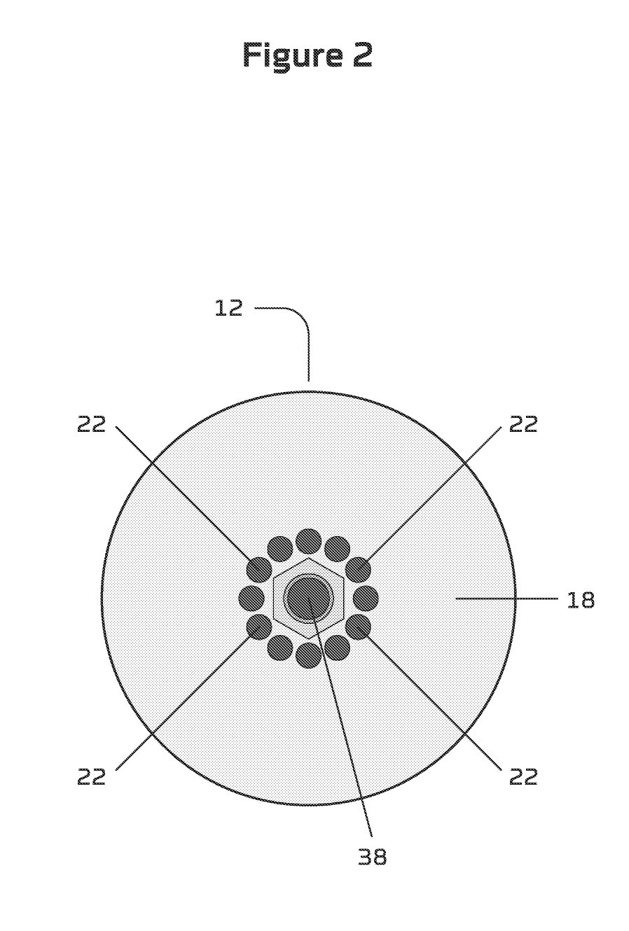

FIG. 2 shows a view of the second surface of the flow distributor 12. There is at least one fluid medium inlet 22 on the second surface 18 adjacent to or at a point 38 opposite the location of the drive shaft 26, or in other words adjacent to the intended centre of rotation of the second surface 18.

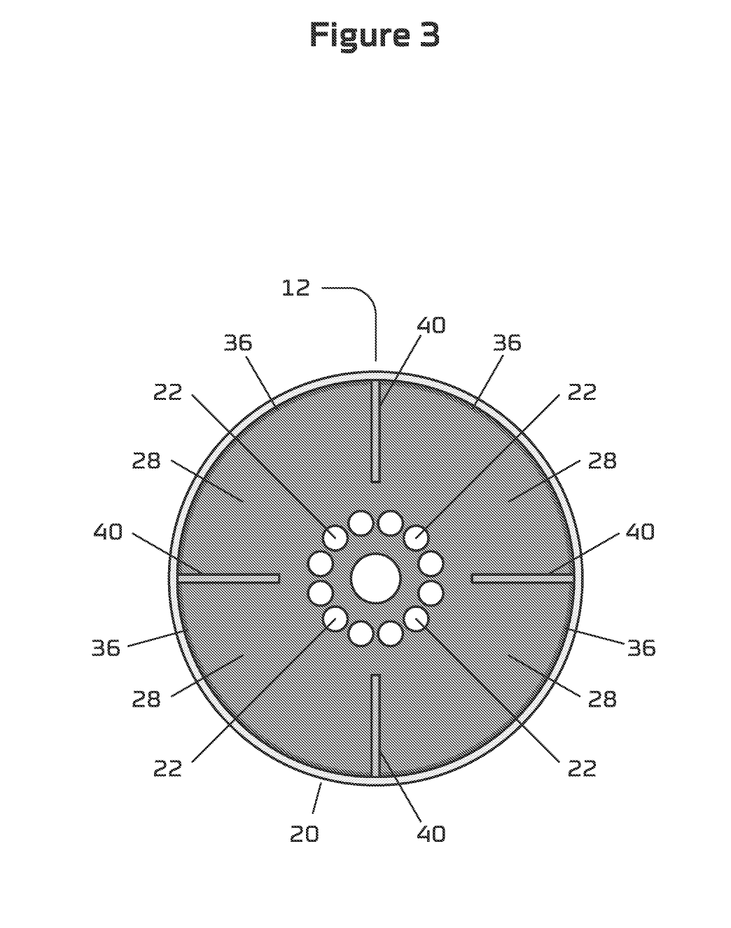

FIG. 3 discloses a cross-sectional view from the first surface of an embodiment the flow distributor 12 shown in FIG. 1 along the line from A to B. In the shown embodiment, there is a plurality of confinements 28 separated from each other by separating walls 40. The confinements 28 may be fully or partially separated from each other. In the shown embodiment, there is a fluid medium inlet 22 in each confinement. However, in case the different confinements are only partially separated from each other, the amount of fluid medium inlets may be smaller than the amount of confinements as the fluid medium and carriers may reach more than one confinement after being transported through such an inlet. In the embodiment shown in FIG. 3 there is also a filtering means 36 along the inner surface of the peripheral wall 20.

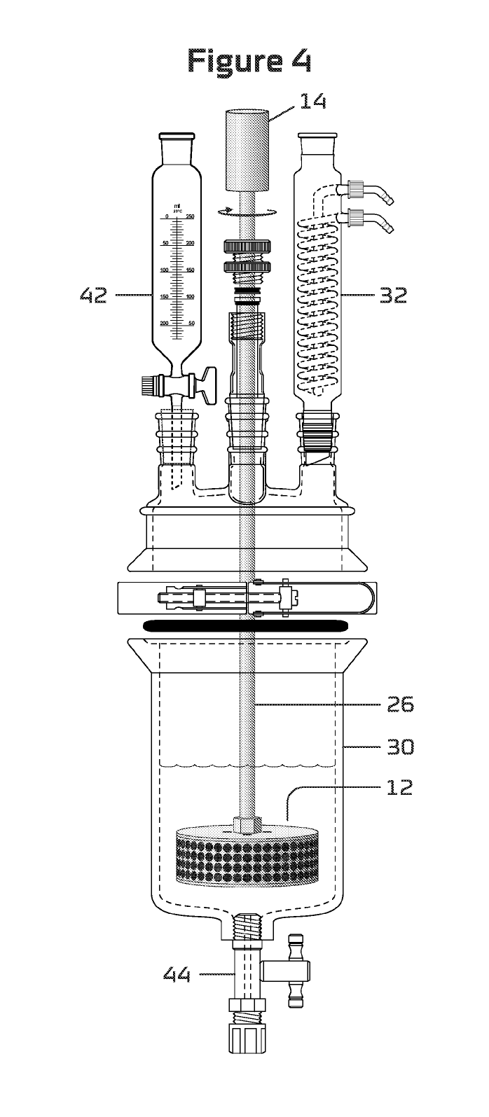

FIG. 4 shows a typical reactor set-up for carrying out the process of the present invention. The process is carried out in reaction vessel, or tank 30. There is an inlet means 42 for adding fluid medium containing starting material and an outlet means 44 for removing fluid medium when the process is finalized. The lid may also be equipped with a reflux cooler 32 and a separate inlet for gaseous substances such as oxygen or hydrogen (not drawn) which can alternatively be added through shaft 26. A transformation device comprising a flow distributor 12 connected to a rotation means 14 is arranged in the reaction vessel 30.

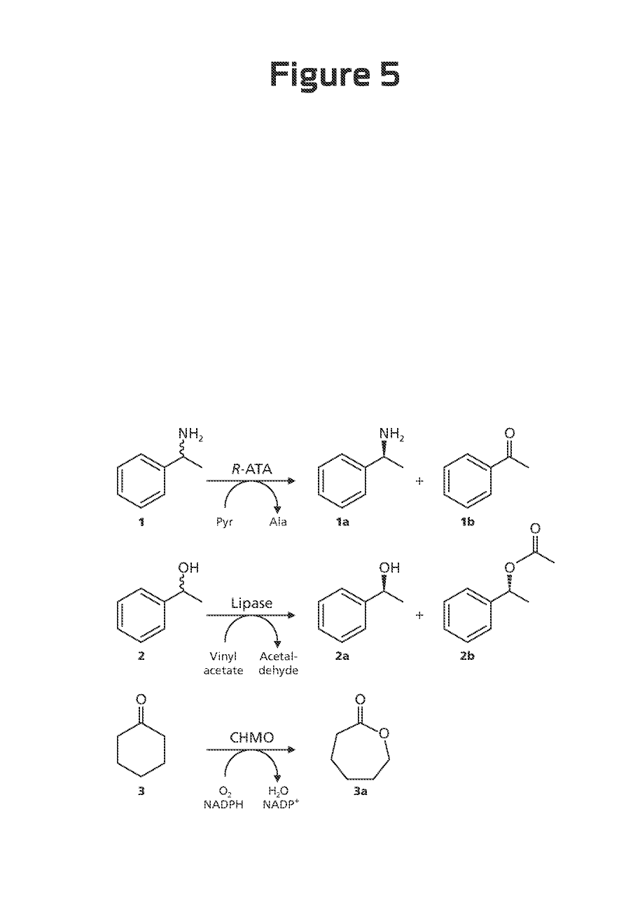

FIG. 5 presents examples of the three biocatalyzed reactions studied in the experimental section. The first reaction is an enzymatic resolution of (R,S)-1-phenylethylamine (referred to as 1) to afford (S)-1-phenyetylamine (referred to as 1a) and acetophenone (referred to as 1b). The reaction is catalyzed by (R)-amine transaminase (referred to as R-ATA) and the reaction further involves transformation of pyruvate (referred to as Pyr) to alanine (referred to as Ala). The second reaction is an enzymatic resolution of (R,S)-1-phenylethanol (referred to as 2) to afford (S)-1-phenylethanol (referred to as 2a) and (R)-1-phenylethyl-acetate (referred to as 2b). The reaction is catalyzed by the immobilized lipase commercially available as Novozyme 435 and vinyl acetate is transformed to acetaldehyde. The third reaction is an enzymatic conversion of cyclohexanone (referred to as 3) to afford ε-caprolactone (referred to as 3a). The reaction is catalyzed by cyclohexanone monooxygenase (referred to as CHMO). During the reaction O₂ and NADPH are transformed to H₂O and NADP⁺.

### Experimental Section

The present invention will be further described by the following examples which are provided for illustration purposes only and are not intended to limit the scope thereof.

All chemicals were either purchased from Fluka (Buchs, CH), Sigma, Merck, VWR or Carl Roth (Karlsruhe, DE) and were used without further purification. Chitosan (deacylation degree >95%, viscosity [1% (w/v) in 1% acetic acid] 500 mPas) was purchased from Heppe Medical Chitosan GmbH (Halle, DE). Novozyme 435 was purchased from Novozymes (Bagsvaerd, DK). All gas chromatography samples were measured on a GC-2010 or GC-2010 Plus from Shimadzu (Kyoto, JP) using columns purchased from Macherey-Nagel (Düren, DE).

### Example 1

### Preparation and Entrapment of Alginate Particles in a Flow Distributor

An alginate solution was prepared by dissolving 3.0 g sodium alginate in 100 ml deionized water. A stock solution of 0.1 M CaCl₂ was also prepared. A Radleys 1000 ml reactor with baffles was furnished with a transformation device comprising the flow distributor S6530 (Nordic ChemQuest AB) mechanically connected to an electrical motor as rotation means. The flow distributor did not contain any inner filter and the fluid medium outlet openings were smaller the alginate particles to be produced. 500 ml 0.1 M CaCl₂ solution was added to the reactor as well as 200 μl detergent solution.

The flow distributor was rotated at a speed of 75 rpm and the alginate solution was added through a 50 ml polypropylene syringe fitted with a 0.7×50 mm stainless steel needle. The solution was added, drop by drop at a high pace (˜2 drops/second). The formed alginate particles had a diameter of about 2-3 mm.

At 75 rpm, the alginate particles were sucked into the flow distributor through the fluid medium inlet but the particles were also able to enter back in the bulk solution through the inlet openings. The rotational speed was increased to 100 and 125 rpm, respectively, but alginate particles were still able to exit the flow distributor at both these rotational speeds.

When the rotational speed was increased to 150 rpm, it was no longer possible for the alginate particles to exit the flow distributor through the inlet openings.

The fluid medium was then drained from the reactor while the rotational speed of the flow distributor was maintained at 150 rpm. The alginate particles were able to exit the flow distributor with the vortex through fluid medium inlet. The experiment was repeated but the rotational speed of the flow distributor was increased to 350 rpm. The alginate particles remained inside the flow distributor when the fluid medium was drained from the reactor.

### Example 2

### Resolution of (R,S)-1-Phenyletylamine to Afford (S)-1-Phenylethylamine Using Immobilized (R)-Transaminase from Gibberella zeae

The biocatalytic reaction that was studied in this example is depicted in scheme 1 of FIG. 5. The abbreviations of scheme 1 are as follows: R-ATA=(R)-amine transaminase, Pyr=pyruvate, and Ala=alanine.

Enzyme production, activity tests and immobilization of the biocatalyst on chitosan support was done following the protocol of Mallin et al., ChemCatChem, 2013, vol. 5, p. 588. 3000 Units of transaminase crude extract were incubated with 2.5 g chitosan support, which was previously activated with glutaraldehyde (1.5% v/v of a 25% solution). Immobilization was done in a volume of 250 ml in sodium phosphate buffer (50 mM, 500 mM NaCl, 0.1 mM pyridoxal-5′-phosphate, pH 7.5) for 16 h (4° C., 20 rpm on orbital shaker). After washing of the immobilized enzyme, a blocking step was newly introduced compared to the recently published protocol. This was done to prevent side reactions of the amines with possibly unreacted aldehyde groups of the activated carrier material. Blocking was performed after immobilization using Tris-HCl buffer (1 M, pH 7.5, 0.1 mM pyridoxal-5′-phosphate) in a volume of 250 ml for 3 h at 4° C. The blocked product was washed three times with 200 ml sodium phosphate buffer (50 mM, pH 7.5, 0.1 mM pyridoxal-5′-phosphate). The initial activity of the immobilized transaminase was determined photometrically and an activity of 278 U/g_{dry} carrier was obtained. From the used starting material of 2.5 g dry chitosan, 29.7 g wet material was received which was directly used for biocatalyses without any drying steps (water content: 91.6%).

The resolution reaction was run in a baffled 1 L-BioFlo 110 fermentor/bioreactor (New Brunswick Scientific) in which a flow distributor S6530 (Nordic ChemQuest AB) mechanically connected to an electrical motor as rotation means had been arranged. 500 ml reaction medium (fluid medium comprising dissolved starting material) consisting of 133 mM (R,S)-1-phenylethylamine and 133 mM pyruvate in sodium phosphate buffer (50 mM, pH 7.5, 0.1 mM pyridoxal-5′-phosphate), and 2.5% DMSO followed by 0.208 g immobilized enzyme was added to the bioreactor. Empty space within the reactor was filled with glass wool to prevent gas entrapment. The temperature of the bioreactor was maintained at 30° C. and the flow distributor was rotated at a speed of 500 rpm. The reaction vessel was closed. The biocatalyst was sucked into the flow distributor.

250 μl samples of the transaminase-catalyzed reaction were taken at regular intervals. 25 μl 10 N sodium hydroxide was added directly to each sample in order to stop any remaining reaction. The samples were then stored at −20° C. until analysis by gas chromatography. Before analysis the samples were thawed and extracted with 500 μl dichloromethane containing 20 mM 2-nonanone as internal standard. After drying of the organic phase with Na₂SO₄, 200 μl of the dichloromethane phase was subsequently derivatized using 20 μl trifluoroacetic acid anhydride for 5 min at room temperature. Then the solvent was evaporated and the residual substance was dissolved in 200 μl fresh dichloromethane. The samples were then analyzed using the following method:

| Column: | Program [r = ° C./min] | Retention times [min] |
|---|---|---|
| Hydrodex | 125° C. 5 min - 20r 150° C. | 2-nonanone: 5.7 |
| γ-TBDAc | 2 min - 0.5r → 160° C. - | Acetophenone: 6.9 |
| | 20r → 180° C. 5 min | (S)-1-phenylethylamine: |
| | | 18.3 |
| | | (R)-1-phenylethylamine: |
| | | 21.0 |

Conversions were determined by calculating the ratio of both enantiomers because only the (R)-enantiomer was converted by the enzyme.

The following conversions were obtained:

| Time (h) | 0 | 1 | 2 | 3 | 4 | 6 |
|---|---|---|---|---|---|---|
| Conversion (%) | 0 | 9 | 17 | 21 | 28 | 37 |

A recycling study was also carried out by re-using the immobilized catalyst in a series of biocatalyses. Between the cycles, the flow distributor was separated from the reaction medium by removal of the flow distributor. The flow distributor was then washed three times by spinning the flow distributor at 500 rpm, 30° C., in a washing solution consisting of sodium phosphate buffer (50 mM, pH 7.5, 0.1 mM pyridoxal-5′-phosphate). Six consecutive biocatalyse batches and intermediate washes were run for the immobilized catalyst in the flow distributor. The conversions after two hours were measured for each batch. The conversion for the first batch was set to 100% relative activity. The following results were obtained:

| 1^{st} batch | 2^{nd} batch | 3^{rd} batch | 4^{th} batch | 5^{th} batch |
|---|---|---|---|---|
| 100% | 100% | 98% | 96% | 94% |

### Example 3

### Resolution of (R,S)-1-Phenylethanol with Immobilized Candida antartica Lipase B

The biocatalytic reaction that was studied is depicted in FIG. 5, scheme 2.

The immobilized enzyme is commercially available as Novozyme 435.

The resolution reaction was run in a baffled 1 L-BioFlo 110 fermentor/bioreactor (New Brunswick Scientific) in which a flow distributor S6530 (Nordic ChemQuest AB) mechanically connected to an electrical motor as rotation means had been arranged. 500 ml reaction medium (fluid medium comprising dissolved starting material) consisting of 1 M vinyl acetate and 1 M rac-1-phenylethanol in n-hexane followed by 2 g dry Novozyme 435 was added to the bioreactor. Empty space within the reactor was filled with glass wool to prevent gas entrapment. The temperature of the bioreactor was maintained at 30° C. and the flow distributor was rotated at a speed of 500 rpm. The reaction vessel was closed. The biocatalyst was sucked into the flow distributor.

50 μl samples of the reaction medium were taken at regular intervals. The samples were diluted directly 1:10 in dichloromethane and stored at −20° C. For gas chromatography analysis, the samples were dried with Na₂SO₄. The non-converted (S)-phenylethanol was used as internal standard. The samples were analyzed using the following parameters:

| Column: | Program [r = ° C./min] | Retention times [min] |
|---|---|---|
| Hydrodex β-3P | 125° C., isothermal 10 min | (R)-phenylethylacetate: |
| | | 6.2 |
| | | (R)-phenylethanol: 7.2 |
| | | (S)-phenylethanol: 7.7 |

Conversion was determined by calculating the ratio of both enantiomers because only the (R)-enantiomer was converted by the enzyme.

The following conversions were obtained:

| | Time (h) | | | | |
|---|---|---|---|---|---|
| | 0 | 1 | 2 | 3 | 4 |
| Conversion | 0% | 18% | 30% | 39% | 45% |

A recycling study was also carried out by re-using the immobilized catalyst in a series of biocatalyses. Between the cycles, the flow distributor was separated from the reaction medium by removal of the flow distributor. The flow distributor was then washed three times by spinning the flow distributor at 500 rpm, in a washing solution consisting of cold acetone. Six consecutive biocatalyse batches and intermediate washes were run for the immobilized catalyst in the flow distributor. The conversions after two hours were measured for each batch. The conversion for the first batch was set to 100% relative activity. The following results were obtained:

| 1^{st} batch | 2^{nd} batch | 3^{rd} batch | 4^{th} batch | 5^{th} batch | 6^{th} batch |
|---|---|---|---|---|---|
| 100% | 89% | 84% | 81% | 65% | 61% |

### Example 4

### Production of ε-Caprolactone from Cyclohexanone Using Calcium

Alginate-Encapsulated Escherichia coli Whole Cells Harboring the Cyclohexanone Monooxygenase from Acinetobacter calcoaceticus NCIMB 9871.

The biocatalytic reaction that was studied is depicted in FIG. 5, scheme 3. Cells expressing cyclohexanone monooxygenase (from now on referred to as CHMO) were obtained by inoculating 400 ml TB medium supplemented with 50 μg/ml kanamycin with 10 ml of an overnight culture (1:40) of E. coli BL21 (DE3) containing a pET28a(+)_CHMO construct (Mallin et al., Enzyme Microb. Technol., 2013, doi: j.enzmictec.2013.01.007) and cultivated until induction at 37° C. The expression of CHMO was induced by 100 μM IPTG at OD₆₀₀ 0.8-1.0 and cultivation was continued for 5-6 h at 30° C. until OD₆₀₀ 7.0-8.0 was reached. Subsequently, the cells were harvested by centrifugation at 4500×g and stored until encapsulation at 4° C.

Prior to encapsulation, the cell pellet was resuspended in Tris-HCl buffer (20 mM, pH 7.5, 1% NaCl, 1% DMSO) and incubated on ice, on an orbital shaker for 30 min for permeabilization. Afterwards, the cells were washed once and resuspended in Tris-HCl buffer (20 mM, pH 7.5, 1% NaCl) at a concentration of 100 g_{wcw}/l. For encapsulation, the suspension was mixed in a 1:1 ratio with alginate solution (3.6%), so that a final concentration of 50 g_{wcw}/l and 1.8% alginate was reached (Zhang et al., Bioprocess Biosyst. Eng., 2010, vol. 33, p. 741). The cell-alginate mixture was passed through a needle (0.8×120 mm) using a membrane pump (Stepdos 03 RC, KNF Flodas) at a flow rate of 6-15 ml/min depending on viscosity into a CaCl₂ solution (0.1 M) on ice and under slow stirring. The formed capsules (average size: 2-3 mm) were hardened for at least 30 min at 4° C. in fresh CaCl₂ solution.

The resolution reaction was run in a baffled 1 L-BioFlo 110 fermentor/bioreactor (New Brunswick Scientific) in which a flow distributor S6530 (Nordic ChemQuest AB) mechanically connected to an electrical motor as rotation means had been arranged. 500 ml reaction medium (fluid medium comprising dissolved starting material) consisting of 20 mM cyclohexanone and 2.5 g/l D-glucose monohydrate in Tris-HCl buffer (20 mM, pH 7.5, 1% NaCl) followed by 20 g wet capsules was added to the bioreactor. Empty space within the reactor was filled with glass wool to prevent gas entrapment. A supply of oxygen of 0.25 liter per minute was arranged. The temperature of the bioreactor was maintained at 25° C. and the flow distributor was rotated at a speed of 500 rpm. The reactor was open under reflux. The biocatalyst was sucked into the flow distributor.

500 μl samples were collected and directly stored at −20° C. to stop the reaction. For gas chromatography analysis, the samples were thawed, extracted with 500 μl dichloromethane containing 2 mM acetophenone as internal standard. Then, the organic phase was dried with Na₂SO₄. The samples were analyzed using the following parameters:

| Column: | Program [r = ° C./min] | Retention times [min] |
|---|---|---|
| Hydrodex β3-3P | 60° C. 10 min - 10r | Cyclohexanone: 13.6 |
| | →160° C. 5 min | Acetophenone: 17.9 |
| | | ε-caprolactone: 20.1 |

The following conversions were obtained:

| Time (h) | 0 | 2 | 4 | 6 | 8 | 17 | 24 |
|---|---|---|---|---|---|---|---|
| Conversion | 0% | 8% | 11% | 17% | 20% | 31% | 36% |

A recycling study was also carried out by re-using the immobilized catalyst in a series of biocatalyses. Between the cycles, the flow distributor was separated from the reaction medium by removal of the flow distributor. The flow distributor was then washed three times by spinning the flow distributor at 500 rpm, in a washing solution consisting of Tris-HCl buffer (20 mM, pH 7.5, 1% NaCl, 10 mM CaCl₂). Six consecutive biocatalyse batches and intermediate washes were run for the immobilized catalyst in the flow distributor. The conversions after two hours were measured for each batch. The conversion for the first batch was set to 100% relative activity. The following results were obtained:

| 1^{st} batch | 2^{nd} batch | 3^{rd} batch | 4^{th} batch | 5^{th} batch | 6^{th} batch |
|---|---|---|---|---|---|
| 100% | 83% | 69% | 59% | 57% | 40% |

### Final Comments

The results obtained in the experimental section shows that the process of the invention provides good and stable results for both recycled and freshly prepared biocatalysts. It also turns out to be very simple to use a flow distributor for managing the biocatalyst. The biocatalyst particles are sucked into the flow distributor at the onset of the reaction. It is also very simple and convenient to wash and recycle the biocatalyst particles.

The person skilled in the art realizes that the present invention by no means is limited to the preferred embodiments described above. On the contrary, many modifications and variations are possible within the scope of the appended claims.

Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

1. A process for chemical and/or biological transformation of at least one starting material dissolved in a fluid medium comprising the steps of:
a) providing a fluid medium containing a dissolved starting material;
b) providing carriers comprising at least one biochemical transformation means selected from the group of an immobilized enzyme and an encapsulated whole cell microorganism or fragments thereof;
c) providing a reactor vessel, in which reactor vessel transformation device has been mounted, said transformation device comprising
a flow distributor having an essentially cylindrical shape, a first essentially flat surface, a second essentially flat surface, and a peripheral wall having an essentially circular cross-section, at least one fluid medium inlet for receiving fluid medium and carriers located at the centre of said second surface, at least one fluid medium outlet permeable for said fluid medium but impermeable for said carriers, said outlet being located on said peripheral wall, a driving shaft located on said first surface for enabling rotation or oscillation of the flow distributor, and at least one confinement wherein said carriers can be trapped and said transformation is performed; and
a means for rotating and/or oscillating the device;
d) adding the fluid medium of step a) and the carriers of step b) to the reactor vessel of step c);
e) rotating said flow distributor using said means at such a rotational speed or oscillatory rotary motion that said fluid medium of step a) and said carriers of step b) are sucked through said at least one fluid medium inlet into said at least one confinement and that said fluid medium is transported out from the flow distributor through said at least one outlet while said carriers remain in said at least one confinement; and
f) maintaining rotating motion of said flow distributor until said transformation is deemed to be completed.

2. A process according to claim 1, wherein said flow distributor comprises a plurality of separate confinements defined by separating walls.

3. A process according to claim 1, further comprising the steps of
g) removing said fluid medium from said reaction vessel while maintaining rotation of said flow distributor at said minimum rotation speed, thereby draining said flow distributor while said carriers are maintained inside said confinement.

4. A process according to claim 1, wherein said carriers are alginate beads encapsulating whole cell microorganisms or fragments thereof.

5. A process according to any of claim 1, 1 wherein said carriers encapsulating whole cell microorganisms or fragments thereof are different native polysaccharides such as straight and branched celluloses, starches, dextrans, agar/agarose, carrageenans, gellan, welan, and xanthan gums, pectins, and chitin/chitosan, and alkylated, acetylated, or glycidylated derivatives thereof; proteins such as collagen, gelatin, and albumin; synthetic polymer gels such as crosslinked poly(acrylamide), polysiloxanes;
thermosresponsive polymers such as poly(/V-isopropyl acrylamide), polyfvinyl caprolactam) and polyfvinyl methyl ether), sol-gel derived carriers prepared by hydrolysis and polycondensation of tetraalkoxysilanes, and porous inorganic carriers such as silica.

6. A process according to claim 4, wherein said fluid medium comprises calcium chloride, and an alginate suspension of whole cell microorganisms or fragments thereof are injected into said fluid medium during step d).

7. A process according to any of claim 4, wherein said whole cell microorganisms are bacteria and/or yeast, or fragments thereof.

8. A process according to claim 7, wherein said microorganisms are chosen from the group of the genera Acetobacter, Achromobacter, Acidovorax, Acinetobacter, Acremonium, Agrobacterium, Alcaligenes, Amycolatopsis, Arthrobacter, Aspergillus, Aureobacterium, Aureobasidium, Bacillus, Beauveria, Brevibacterium, Burkholderia, Caldariomyces, Candida, Chromobacterium, Clonostachys, Clostridia, Comamonas, Coprinus, Corynebacterium, Corynesporium, Cryptococcus, Curvularia, Enterobacter, Erwinia, Escherichia, Fusarium, Geotrichum, Gluconobacter, Gordonae, Haloferax, Helm inthosporium, Hum icola, Klebsiella, Kluyveromyces, Lactobacillus, Leptoxyphium, Leuconostoc, Microbacterium, Mortierella, Mucor, Mycobacterium, Neurospora, Nocardia, Ochrobactrum, Penicillium, Pichia, Plantomycetes, Protaminobacter, Pseudomonas, Pyrococcus, Rhizopus, Rhodococcus, Rhodosporidium, Rhodotorula, Rubiginosus, Saccharomyces, Serratia, Shigella, Spirulina, Staphylococcus, Stenotrophomonas, Streptomyces, Sulfolobus, Thermoactinomyces, Thermoanaerobacter, Thermoanaerobium, Thermobifida, Thermomyces, Thermus, Trigonopsis, Vibrio, Yarrowia, Zygosaccharomyces, and Zymomonas, or combinations thereof. Cells trapped and utilized according to the method disclosed in the present invention could also be derived from plants (for instance Arabidopsis, Hevea, Geranium, or Prunus) or animals, including humans.

9. A process according to any of claim 1, wherein the biochemical transformation means is an immobilized enzyme selected from the group of oxidoreductases, transferases, hydrolases, lyases, isomerases, and ligases.

10. A process according to claim 1, wherein the enzyme is immobilized on a carrier selected from the group of different native polysaccharides such as straight and branched celluloses, starches dextrans, agar/agarose, carrageenans, gellan, wellan, and xanthan gums, pectins, and chitin/chitosan, and alkylated, acetylated, or glycidylated derivates thereof, proteins such as collagen, gelatin, and albumin, synthetic polymer gels such as cross-linked poly(acrylamide), polysiloxanes, thermosresponsive polymers such as poly(/V-isopropyl acrylamide), polyvinyl caprolactam) and polyvinyl methyl ether), sol-gel-derived carriers prepared by hydrolysis and polycondensation of tetraalkoxysilanes, polystyrene, polyacrylates, polymethacrylates, polyamides, polyvinyl aziactone), vinyl and allyl polymers, benthonite, zeolite, diatomaceous earth, carbon, silica, glass (non porous and controlled pore), metals, and controlled pore metal such as alumina, zirconia and titania.

11. Use of a transformation device comprising a flow distributor having an essentially cylindrical shape, a first essentially flat surface, a second essentially flat surface, and a peripheral wall having an essentially circular cross-section, at least one fluid medium inlet for receiving fluid medium and carriers located at the centre of said second surface, at least one fluid medium outlet permeable for said fluid medium but impermeable for said carriers, said outlet being located on said peripheral wall, a driving shaft located on the first surface for enabling rotation or oscillation of the flow distributor, and at least one confinement wherein said carriers can be trapped and said transformation is performed; and
a means for rotating and/or oscillating the device;
in a process in accordance with any claim 1.

12. Use according to claim 11, wherein said flow distributor comprises a plurality of separate confinements defined by separating walls.

13. Use of a flow distributor having an essentially cylindrical shape, a first essentially flat surface, a second essentially flat surface, and a peripheral wall having an essentially circular cross-section, at least one fluid medium inlet for receiving fluid medium and carriers located at the centre of said second surface, at least one fluid medium outlet permeable for said fluid medium but impermeable for said carriers, said outlet being located on said peripheral wall, a driving shaft located on the first surface for enabling rotation and/or oscillation of the flow distributor, and at least one confinement wherein said carriers can be trapped and said transformation is performed, in a process in accordance with claim 1.

14. Use according to claim 13, wherein said flow distributor comprises a plurality of separate confinements defined by separating walls.
